(19) United States
(12) Patent Application Publication
(10) Pub. No.: US 2026/0167586 A1
(43) Pub. Date: Jun. 18, 2026

(54) CONVERSION OF UNSATURATED HYDROCARBON CONTAINING OFF-GASES FOR MORE EFFICIENT HYDROCARBON PRODUCTION PLANT

(71) Applicant: TOPSOE A/S, Kgs. Lyngby (DK)

(72) Inventors: Sudip DE SARKAR, Kokkedal (DK); Thomas Sandahl CHRISTENSEN, Kgs. Lyngby (DK); Kim AASBERG-PETERSEN, Allerød (DK)

(73) Assignee: TOPSOE A/S, Kgs. Lyngby (DK)

(21) Appl. No.: 19/123,936

(22) PCT Filed: Nov. 2, 2023
(86) PCT No.: PCT/EP2023/080607

§ 371 Date: Apr. 24, 2025

(30) Foreign Application Priority Data
Nov. 4, 2022 (DK) PA202200998

(51) Int. Cl. C07C 1/12

(52) U.S. Cl. CPC C07C 1/12

(57) ABSTRACT

The present invention relates to a more efficient, sustainable system and process, wherein an off-gas comprising unsaturated hydrocarbons from a synthesis stage is recycled to the syngas stage through an off-gas conversion stage comprising hydrogenation of unsaturated hydrocarbons in said off-gas. The exotherm across the adiabatic hydrogenation unit is controlled efficiently by using dilution gas.

### TECHNICAL FIELD

The present invention relates to a more efficient sustainable hydrocarbon production system and process, where the off-gas from a synthesis stage is recycled to a syngas stage and where the exotherm in the off-gas conversion stage is controlled.

### BACKGROUND

Carbon capture and utilization (CCU) has gained more relevance in the light of the rise of atmospheric CO₂ since the Industrial Revolution. In one way of utilizing CO₂, CO₂ and H₂ can be converted to synthesis gas (a gas rich in CO and H₂), which can be converted further to valuable products including eFuels such as jet-fuel, kerosene such as synthetic paraffinic kerosene (SPK) and/or diesel produced for example by the Fischer-Tropsch (F-T) process. Another application could also be production of eChemicals, such as -olefins from syngas.

Existing technologies apply the reverse water gas shift (RWGS) processes to convert CO₂ and H₂ to synthesis gas. The reverse water gas shift reaction proceeds according to the following reaction:

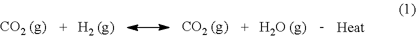

In a system such as a hydrocarbon production plant, reaction (1) can take place in a syngas generation stage. Reaction (1) is an endothermic reaction thus high temperatures are needed to obtain sufficient conversion of carbon dioxide into carbon monoxide to make the process economically feasible. The synthesis gas can subsequently be converted to a raw hydrocarbon stream in a downstream process, e.g. a Fischer-Tropsch (F-T) process, which through a series of chemical reactions converts CO and H₂ to liquid hydrocarbons. In a system such as a chemical production plant, the F-T process can proceed in a F-T synthesis stage. In another type of plant, an eFuel plant, CO₂ and H₂ feeds can be converted to methanol followed by conversion of methanol to kerosene or diesel. Alternatively, the plant can also be an olefin production plant, such as ethylene, propylene etc. where methanol from CO₂ and H₂ feeds are converted to olefin product.

Irrespective of the application, all of these processes are associated with formation of by-product off-gas streams. Reuse of these by-product off-gas streams to produce more syngas is essential for overall efficiency and feasibility of such processes.

In many instances, off-gas streams contain unsaturated hydrocarbons, such as olefins or alkenes (CₙH₂ₙ; n≥2). Conversion of said off-gas streams, containing unsaturated hydrocarbons, requires hydrogenation (i.e., conversion of unsaturated hydrocarbons to saturated hydrocarbons (CₙH₂ₙ₊₂; n≥2)) as shown below

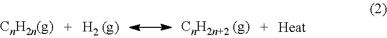

Hydrogenation of unsaturated hydrocarbons is an essential step to avoid possible C-formation in syngas generation step using for example Ni-based catalysts. Moreover, hydrogenation reactions are exothermic. To ensure high conversion of unsaturated hydrocarbons, the exotherm needs to be controlled. As described in U.S. Pat. No. 9,162,886B2, this can be done in the catalyst bed of an adiabatic hydrogenation reactor, where reactant gas enters at 70-120° C. and causes an exotherm of 50-90° C. Alternatively, a cooled reactor operating at a temperature range preferably within 120-150° C. can also be used. The cooled reactor can be for example a boiling water reactor (BWR), which is a special reactor requiring relatively more complicated design and associated additional equipment. For example, in a BWR typically the reactant gas is passed through the catalyst-filled tubes which exchange heat with boiling water on the shell side of the reactor, producing steam. This type of reactor requires steam drum, risers, downcomers etc. Cooled reactors can also use a gaseous stream in the shell side instead of boiling water reactor.

The higher the olefin content in the off-gas stream, the higher the exotherm across the adiabatic bed causing inefficient hydrogenation of unsaturated hydrocarbons. To control the exotherm in an adiabatic bed, circulation of a part of the hydrogenation effluent can be performed. However, this solution requires at least a recycle effluent cooler and effluent recycle compressor or blower. Thus, traditionally, an off-gas stream with high olefin content would require use of either a cooled hydrogenation reactor solution or an effluent recycle solution. Both options are more complicated and require more equipment than a simple adiabatic bed.

A need therefore exists for a simple and effective system and process for off-gas recycle in such systems/processes.

### SUMMARY

It has been found by the present inventor(s) that the exotherm in the off-gas stream can be controlled within a system for producing a product (e.g. hydrocarbon product) stream, said system comprising:
a first feed comprising carbon dioxide;
a second feed comprising hydrogen;
optionally, an external dilution feed;
a syngas stage arranged to receive at least a first portion of said first feed, at least a first portion of said second feed, and at least a portion of a pre-converted off-gas stream, and to provide a first synthesis gas stream;
a synthesis stage arranged to receive said first synthesis gas stream, and to provide one or more product stream(s) and an off-gas stream comprising unsaturated hydrocarbons;
mixing means arranged to combine at least a portion of the off-gas stream with a dilution feed and provide a combined feed;
an off-gas conversion stage arranged to receive at least a portion of the combined feed and provide a pre-converted off-gas stream to the syngas stage;
   wherein the off-gas conversion stage comprises at least one hydrogenation unit arranged to convert unsaturated hydrocarbons in said combined feed to saturated hydrocarbons in said pre-converted off-gas stream;
   and wherein the dilution feed is one or more feeds selected from:
a second portion of said first feed of carbon dioxide,
a second portion of said second feed of hydrogen,
an external dilution feed, comprising a carbon dioxide-rich feed, a steam feed, a methane-rich feed, a hydrogen-rich feed, or a combination thereof.

Exotherm development in the present system is controlled by means of the dilution feed.

A process for production of a product stream in a system according to the invention is also provided, said process comprising the steps of:
providing a first feed comprising carbon dioxide, a second feed comprising hydrogen, and optionally, an external dilution feed;
feeding at least a first portion of said first feed, at least a first portion of said second feed and at least a portion of a pre-converted off-gas stream to the syngas stage to provide a first synthesis gas stream;
feeding at least a portion of the first synthesis gas stream to the synthesis stage to provide one or more product stream(s) and the off-gas stream comprising unsaturated hydrocarbons;
combining at least a portion of the off-gas stream with a dilution feed in said mixing means to provide a combined feed;
feeding at least a portion of the combined feed to the off-gas conversion stage to provide a pre-converted off-gas stream to the syngas stage; and
converting unsaturated hydrocarbons in said combined feed to saturated hydrocarbons in said pre-converted off-gas stream;
and wherein the dilution feed is one or more feeds selected from:
a second portion of said first feed of carbon dioxide,
a second portion of said second feed of hydrogen,
an external dilution feed, comprising a carbon dioxide-rich feed, a steam feed, a methane-rich feed, a hydrogen-rich feed, or a combination thereof.

The present system and process thus provide a controlled hydrogenation pathway.

The effects and advantages are, inter alia:
1. The exotherm in the hydrogenation reactor is controlled without additional complicated equipment. Only an adiabatic hydrogenation reactor will be needed.
2. Addition of the dilution feed will function as heat sink with no or minor participation in hydrogenation, resulting in the control of an exotherm.
3. When at least a part of the first feed comprising carbon dioxide is used as dilution feed, it provides perfect synergy to the process as dilution feed, in this case, CO₂ gets reacted in subsequent steps ofthe syngas stage.

Further details of the technology are provided in the enclosed dependent claims, figures and examples.

### LEGENDS TO THE FIGURES

The technology is illustrated by means of the following schematic illustrations, in which:

FIG. 1 shows a simple layout of one aspect of the system of the invention.

FIG. 2 shows a more developed layout of the system of the invention.

### DETAILED DISCLOSURE

Unless otherwise specified, any given percentages for gas content are % by volume. All feeds are preheated as required.

A “stage” comprises one or more “units” which perform a change in the chemical composition of a feed, and may additionally comprise elements such as e.g. heat exchanger, mixer or compressor, which do not change the chemical composition of a feed or stream.

The term “synthesis gas” (abbreviated to “syngas”) is meant to denote a gas comprising hydrogen, carbon monoxide, carbon dioxide and small amounts of other gasses, such as argon, nitrogen, methane, steam, etc.

In a first aspect, a system for producing a raw hydrocarbon stream is provided. The system comprises:
a first feed comprising carbon dioxide;
a second feed comprising hydrogen;
optionally, an external dilution feed;
a syngas stage arranged to receive at least a first portion of said first feed, at least a first portion of said second feed, and at least a portion of a pre-converted off-gas stream, and to provide a first synthesis gas stream;
a synthesis stage arranged to receive said first synthesis gas stream, and to provide one or more product stream(s) and an off-gas stream comprising unsaturated hydrocarbons;
mixing means arranged to combine at least a portion of the off-gas stream with a dilution feed and provide a combined feed;
an off-gas conversion section arranged to receive at least a portion of the combined feed and provide a pre-converted off-gas stream to the syngas stage;
   wherein the off-gas conversion section comprises at least one hydrogenation unit arranged to convert unsaturated hydrocarbons in said combined feed to saturated hydrocarbons in said pre-converted off-gas stream;
   and wherein the dilution feed is one or more feeds selected from:
a second portion of said first feed of carbon dioxide,
a second portion of said second feed of hydrogen,
an external dilution feed, comprising a carbon dioxide-rich feed, a steam feed, a saturated hydrocarbon feed, a hydrogen-rich feed, or a combination thereof.

### Feeds

A first feed comprising carbon dioxide is provided, of which at least a first portion is provided to the syngas stage. Suitably, the first feed consists essentially of CO₂. The first feed of CO₂ is suitably “CO₂ rich” meaning that the major portion of this feed is CO₂; i.e. over 75%, such as over 85%, preferably over 90%, more preferably over 95%, even more preferably over 99% of this feed is CO₂. One source of the first feed of carbon dioxide can be one or more exhaust stream(s) from one or more chemical plant(s). One source of the first feed of carbon dioxide can also be carbon dioxide captured from one or more process stream(s) or atmospheric air. Another source of the first feed could be CO₂ captured or recovered from the flue gas for example from fired heaters, steam reformers, and/or power plants. The first feed may in addition to CO₂ comprise for example steam, oxygen, nitrogen, oxygenates, amines, ammonia, carbon monoxide, and/or hydrocarbons.

A second feed comprising hydrogen is provided, of which at least a first portion is provided to the syngas stage. Suitably, the second feed consists essentially of hydrogen. The second feed of hydrogen is suitably “hydrogen rich” meaning that the major portion of this feed is hydrogen, i.e. over 75%, such as over 85%, preferably over 90%, more preferably over 95%, even more preferably over 99% of this feed is hydrogen. One source of the second feed of hydrogen can be one or more electrolyser units. In addition to hydrogen the second feed may for example comprise steam, nitrogen, argon, carbon monoxide, carbon dioxide, and/or hydrocarbons. In some cases, a minor content of oxygen may be present in this feed, typically less than 100 ppm.

The ratio of H₂/CO₂ provided to the syngas stage inlet varies from 2.0-7.0. This ratio is defined as any H₂ and CO₂ in external streams (i.e. not including hydrogen and/or carbon dioxide via recycled off-gas streams). This ratio will depend upon the desired end-product in the synthesis stage. For example, the desired H₂/CO-ratio of the synthesis gas will typically be around 2.0 if it is to be used in a Fischer-Tropsch synthesis. For an F-T synthesis stage the H₂/CO₂-ratio at the system inlet (i.e. not including hydrogen and/or carbon dioxide in any recycle streams) should be in the range of 2.0-7.0 or more preferably from 3.0-6.0 and most preferably 3.0-5.0.

An external dilution feed is optionally provided to the plant, and will be discussed in detail below.

### Additional Feeds

Dependent on the specific choice of reactors/catalysts within the system, additional feeds might be required such as to the syngas stage and/or the synthesis stage. In addition, steam feeds may be added as required.

In one aspect, the syngas stage comprises an autothermal reactor (ATR) section and a fifth feed comprising an oxidant is provided to the syngas stage. Suitably, the fifth feed consists essentially of oxygen. The fifth feed of O₂ is suitably “O₂ rich” meaning that the major portion of this feed is O₂; i.e. over 75% such as over 90% or over 95%, such as over 99% of this feed is O₂. This fifth feed may also comprise other components such as nitrogen, argon, CO₂, and/or steam. This fifth feed will typically include a minor amount of steam (e.g. 5-10%). The source of oxygen can be at least one air separation unit (ASU) and/or at least one membrane unit. The source of oxygen can also be at least one electrolyser unit. A part or all of fifth feed may come from at least one electrolyser. Steam may be added to the fifth feed comprising oxygen, upstream the ATR unit.

In one aspect, a sixth feed comprising hydrocarbons is arranged to be feed to the syngas stage, upstream an ATR unit. This sixth feed may additionally comprise other components such as CO₂ and/or CO and/or H₂ and/or steam and/or other components such as nitrogen and/or argon. This feed may be a natural gas feed. Suitably, the sixth feed consists essentially of hydrocarbons. The sixth feed of hydrocarbons is suitably “hydrocarbon rich” meaning that the major portion of this feed is hydrocarbons, i.e. over 50%, e.g. over 75%, such as over 85%, preferably over 90%, more preferably over 95%, even more preferably over 99% of this feed is hydrocarbons. The concentration of hydrocarbons in this sixth feed is determined prior to steam addition (i.e. determined as “dry concentration”).

In one aspect, the system does not comprise an external hydrocarbon feed such as a natural gas feed. In this way, the only source of carbon, fed to the syngas stage, comes from the first feed of carbon dioxide, the second feed of hydrogen and/or from at least one recycle stream from the synthesis stage.

### Syngas Stage

The syngas stage is arranged to receive at least a first portion of said first feed, and at least a first portion of said second feed, and at least a portion of pre-converted off-gas feed from synthesis stage to provide a first synthesis gas stream. Optionally, said first and second feeds are arranged to be fed to the syngas stage as an admixed feed. The term “provide a synthesis gas stream” in this context must be understood as to “produce a synthesis gas stream”.

In one aspect, the primary unit of the syngas stage comprises a stand-alone reverse water gas shift (RWGS) unit, wherein the RWGS unit comprises at least one reverse water gas shift (RWGS) reactor. Within the RWGS reactor, reverse water gas shift (RWGS) processes convert CO₂ and H₂ to synthesis gas as given by reaction (1). The RWGS reaction (1) is an endothermic process which requires significant energy input for the desired conversion. In traditional reactors, heated combustion of, for example natural gas or other combustibles, provide the energy. Alternatively, the RWGS reactor may be an e-RWGS reactor, in which energy is provided by electrical heating.

In parallel to the reverse water gas shift reaction, methanation may also take place according to one or both methanation reactions in the presence of a non-selective RWGS catalyst:

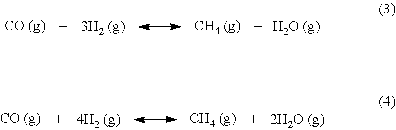

Reaction (3) and (4) are typically at or close to equilibrium at the reactor outlet.

Higher hydrocarbons may cause carbon formation within the RWGS unit. Thus, in one aspect, the hydrogenated off-gas stream is fed to an additional reactor such that the hydrocarbons are removed before reintroducing the hydrogenated off-gas into, and in one aspect directly into, the syngas stage comprising a RWGS unit. This allows the syngas stage to comprise any type of stand-alone RWGS unit. Another option is to feed the hydrogenated off-gas stream to a Water Gas Shift reactor, followed by an adiabatic prereforming reactor.

In one aspect, the stand-alone RWGS unit comprises a RWGS reactor in which the reactor comprises a catalyst with activity for steam reforming i.e. the reverse of reaction (3) and (4). In the following the wording “selective RWGS” shall mean that only the reverse water gas shift reaction takes place either on a catalyst or in a reactor while “non-selective RWGS” shall mean that other reactions such as one or more of the methanation reactions (including also reverse methanation) takes place in addition to reverse water gas shift.

In one aspect the syngas stage, in addition to a RWGS stage, comprises a reforming unit. In one aspect a RWGS stage is arranged upstream a reforming stage, which suitably includes an autothermal reactor (ATR) section.

In one aspect the syngas stage primarily comprises a RWGS stage, which suitably includes an autothermal reactor (ATR).

In the following, preferred aspects of the syngas stage are provided.

Syngas Stage Comprising an e-RWGS Unit

In one aspect, the syngas stage comprises an electrically heated reverse water gas shift (e-RWGS) unit, wherein the e-RWGS unit comprises one or more e-RWGS reactor(s). Electrically-heated reverse water gas shift (e-RWGS) uses an electric resistance-heated reactor to perform a more efficient reverse water gas shift process (reaction 1) and substantially reduces or preferably avoids the use of fossil fuels as a heat source. More details of e-RWGS units and a full description can be found in the art such as in WO22079098 A1, which is hereby incorporated by reference.

In case of non-selective e-RWGS, methanation according to reactions (3) and/or (4) takes place in addition to the RWGS reaction. This has the advantage that the concentration of carbon monoxide internally in the reactor is lower than if only RWGS takes place. This is especially important in the low to moderate temperature range up to ca. 600-800° C. In this temperature range, a potential for carbon formation or metal dusting exists or is significantly larger with a selective RWGS catalyst than with a non-selective catalyst, cf. the carbon formation reactions:

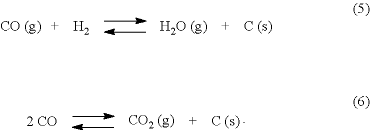

In one aspect, when using a non-selective e-RWGS reactor, the methane concentration by volume in the gas leaving the e-RWGS reactor is lower than 6% such as lower than 4% or preferably less than 3%. High product gas temperature ensures that the final synthesis gas has low methane concentration, despite the methane concentration having a peak somewhere along the reaction zone. Therefore, this reactor configuration can be operated with none, or little, methane in the feed and only little methane in the final synthesis gas.

It is advantageous in most cases that the concentration of methane in the synthesis gas is as low as possible, as methane does not act as a reactant in the downstream synthesis stage.

In one aspect the maximum methane concentration in the e-RWGS unit is higher than both the concentration of the inlet gas to the e-RWGS unit and the concentration of the effluent gas from the e-RWGS unit.

The e-RWGS unit comprises one or more e-RWGS reactors, and in one aspect, consists of a single e-RWGS reactor.

A high exit temperature has the further advantage that a higher conversion of CO₂ into CO. In an aspect the exit temperature of the gas from the e-RWGS reactor is higher 900° C., such than higher than 1000° C. or even higher than 1050° C. It is an advantage of this reactor that a higher temperature can be achieved than what is typically possible with an externally fired reactor.

Another means to have a low methane concentration at the exit of the e-RWGS reactor is to have a low to moderate pressure, such as between 5 and 20 bars or between 8 and 12 bars. In this aspect the gas leaving the e-RWGS unit will typically be cooled, and water will be (partially) removed by condensation followed by compression to the desired pressure for downstream applications.

### Syngas Stage Comprising Autothermal Reactor Unit

In one aspect, the syngas stage comprises a an autothermal reactor (ATR) section. An ATR typically comprises a burner, a combustion chamber, and a catalyst bed contained within a refractory lined pressure shell. In an ATR, partial combustion of hydrocarbons by sub-stoichiometric amounts of oxygen is followed by steam reforming—i.e., the reverse of reaction (3) and (4)—of the partially combusted hydrocarbons in a fixed bed of steam reforming catalyst. Typically, the gas is at or close to equilibrium at the outlet of the reactor with respect to steam reforming and water gas shift reactions. More details of ATR and a full description can be found in the art such as “Studies in Surface Science and Catalysis, Vol. 152” Synthesis gas production for FTsynthesis”; Chapter 4, p.258-352, 2004”.”.

In one aspect, the syngas stage comprises a RWGS unit and an ATR unit such that a RWGS unit is followed by an ATR unit. In aspects with an ATR downstream an RWGS reactor, the effluent gas from the RWGS reactor is directed to an autothermal reactor. Within the ATR, the effluent gas from the RWGS reactor reacts with an oxidant such as oxygen to produce the final synthesis gas. The gas leaving the RWGS reactor is preferably not cooled (except for heat loss and by mixing with other streams). Cooling of the gas increases the oxygen consumption in the ATR.

In one aspect a feed gas comprising hydrocarbons is added to the effluent gas from the RWGS reactor upstream of the ATR. Thus, in one aspect, higher hydrocarbons are not removed from the hydrogenated off-gas stream before it is recycled into the syngas stage. In one aspect, an additional feed gas comprising hydrocarbons such as a feed described by the sixth feed is added to the ATR. Part or all of the sixth feed may be desulfurized and prereformed before the inlet of the ATR unit. All feeds are preheated as required.

In one aspect the syngas stage consists of a reforming unit, which suitably includes an autothermal reformer (ATR) unit. Within this aspect, the first feed, the second feed, a fifth feed comprising an oxidant such as oxygen, and a feed gas comprising hydrocarbons such as the hydrogenated off-gas from the synthesis stage are directed to the ATR. In one aspect, an additional feed gas comprising hydrocarbons such as a feed described by the sixth feed is directed to the ATR.

Typically, the effluent gas stream from the ATR reactor i.e. the synthesis gas stream has a temperature of 900-1100° C. The synthesis gas normally comprises hydrogen, carbon monoxide, carbon dioxide, and steam. Other components such as methane, nitrogen, and argon may also be present often in minor amounts. The operating pressure of the ATR reactor will be between 5 and 100 bars or more preferably between 15 and 60 bars. The synthesis gas stream from the ATR may be cooled in a cooling train normally comprising a waste heat boiler(s) (WHB) and one or more additional heat exchangers. The cooling medium in the WHB is (boiler feed) water which is evaporated to steam. The synthesis gas stream is further cooled to below the dew point for example by preheating the utilities and/or partial preheating of one or more feed streams and cooling in air cooler and/or water cooler. Condensed H₂O is taken out as process condensate in a separator to provide a synthesis gas stream with low H₂O content, which is sent to the synthesis stage.

In one preferred aspect an e-RWGS unit is followed by a reforming unit, which suitably includes an autothermal reformer (ATR). Within this aspect, the final synthesis gas typically has a temperature above 950° C., such as above 1020° C., or 1050° C. or above. In this particular aspect the exit temperature from the e-RWGS reactor will typically be between 600-900° C. such as between 700-850° C. The e-RWGS reactor may in this aspect either be selective or preferably be non-selective.

In aspects with an ATR downstream an e-RWGS unit comprising a non-selective RWGS reactor, the methane concentration leaving the RWGS reactor will preferably be lean, such as less than 20% or preferably less than 12%. A relatively low concentration has the advantage that less oxidant is needed in the ATR.

In aspects with an ATR downstream an e-RWGS unit, an advantage is that the power needed for the e-RWGS reactor is reduced due to the lower exit temperature. In one aspects, part or all of the oxygen generated by electrolysis of steam to produce hydrogen for the e-RWGS reactor is used in the ATR.

By use of an e-RWGS unit (as compared to a regular, fired RWGS unit), it is possible to produce a product gas with low content of CO₂, which is desired for some applications, e.g. F-T synthesis, since the high temperature of e-RWGS operation ensures a high conversion of CO₂ to CO.

The “ATR unit” may be a partial oxidation “POX” unit. A POX unit is similar to an ATR unit except for the fact that the ATR reactor is replaced by a POX reactor. The POX rector generally comprises a burner and a combustion chamber contained in a refractory lined pressure shell.

The ATR unit could also be a catalytic partial oxidation (cPOX) unit.

### Synthesis Stage

A synthesis stage is arranged to receive the first synthesis gas stream (from the syngas stage), and to provide one or more product stream(s) and an off-gas stream comprising unsaturated hydrocarbons. In particular, the product stream may be a hydrocarbon product stream. Various synthesis stages are described below.

### Fischer-Tropsch Synthesis Stage

In one preferred embodiment, the synthesis stage is a Fischer-Tropsch (F-T) synthesis stage. The Fischer-Tropsch (F-T) synthesis stage receives said first synthesis gas stream and provides a raw hydrocarbon stream and an off-gas stream comprising unsaturated hydrocarbons.

The off-gas produced in the F-T process, comprises higher hydrocarbons including olefins, CO, CO₂, H₂, CH₄ and inert gases (N₂, Ar etc.). The exact composition of the off-gas may vary significantly depending on the process conditions and catalyst used in the F-T synthesis stage. A key parameter for making the above utilization of CO₂ sustainable is to recycle the off-gas such that the carbon therein may be reintroduced in the production of the synthesis gas, thereby improving the overall carbon efficiency of the process.

One of the challenges of using an off-gas comprising unsaturated hydrocarbons in the syngas stage is that it may lead to potential carbon formation in the syngas stage. Therefore, it is preferred to convert the unsaturated hydrocarbons into alkanes in a hydrogenation process followed by removal of the higher hydrocarbons. Hydrogenation is an exothermic reaction and thus, the extent of exotherm depends to a great extent on the olefin content in the off-gas. Lack of control over the exotherm can lead to extensive carbon formation.

At the inlet of said F-T synthesis stage, the synthesis gas stream suitably has a H₂/CO ratio in the range 1.00-4.00; preferably in the range 1.50-2.10. In another aspect, the synthesis gas stream at the inlet of said F-T synthesis stage suitably has a (H₂— CO₂)/(CO+CO₂) ratio in the range 1.50-2.50; preferably 1.80-2.30, more preferably 1.90-2.20.

The product stream provided by the F-T synthesis stage is a raw hydrocarbon stream comprising higher hydrocarbons such as long chain hydrocarbons and olefins. The ratio between long chain hydrocarbons and olefins in the raw product from the F-T synthesis stage depends on the type of catalyst, reaction temperature etc. used in the process.

The hydrocarbon-containing F-T off-gas stream is produced as side product. The F-T off-gas stream typically comprises carbon monoxide (5-40 vol. %), hydrogen (10-50 vol %), carbon dioxide (20-50 vol %), methane (10-40 vol %) and higher hydrocarbons (1-20 vol %).

Additional components such as argon and nitrogen may also be present in smaller amounts. The higher hydrocarbons comprise olefins and paraffins with two or more carbon atoms. The olefin content can be >0.3% or >3 mol % or even >5 mol % or even >10 mol % but olefin content is <15 mol % In one aspect, the hydrogenated off-gas from the off-gas conversion stage is arranged to be fed as feed to the e-RWGS unit after higher hydrocarbons have been removed.

### Other Synthesis Stages

In one preferred embodiment, the synthesis stage is a Methanol-to-Olefin (MTO) synthesis stage in which synthesis gas is first converted to a first methanol stream, followed by a purification stage where said first methanol stage is partially purified to obtain a second methanol product stream, which is further converted to olefins such as ethylene and propylene. The off-gas stream(s) comprising unsaturated hydrocarbons from MTO synthesis is hydrogenated before sending it to syngas stage for production of first synthesis gas stream.

In another embodiment, the synthesis stage is a is a Methanol-to-Jet (MTJ) synthesis stage in which synthesis gas is first converted to a first methanol stream, followed by a purification stage where said first methanol stream is partially purified to obtain a second methanol product stream, which is further converted to jet fuel, optionally intermediate production of olefins. The off-gas stream(s) comprising unsaturated hydrocarbons from MTJ synthesis is hydrogenated before sending it to syngas stage for production of first synthesis gas stream.

### Controlled Hydrogenation

Mixing means is arranged to combine at least a portion of the off-gas from the synthesis stage with a dilution feed and provide a combined feed. The concentration of olefins in the combined feed is thereby lowered compared to the olefin concentration in the off-gas. The combined feed is fed to an off-gas conversion stage, wherein the combined feed is hydrogenated. In this way, the dilution feed is used as a source of heat sink in hydrogenation, resulting in efficient control of the exotherm.

In one aspect, the off-gas has an olefin content of preferably >3 mol %; more preferably >4 mol %, even more preferably >5 mol % but suitably <15 mol %.

Mixing means can comprise pipe connectors such as T-connectors or Y-connectors, or any combination of elements suitable for mixing two gas streams.

### Dilution Feed

As above, mixing means is arranged to combine at least a portion of the off-gas stream with a dilution feed and provide a combined feed. To provide the pre-converted off-gas stream, the system and process therefore requires at least one dilution feed.

The dilution feed is one or more feeds selected from:
a second portion of said first feed of carbon dioxide,
a second portion of said second feed of hydrogen,
an external dilution feed, comprising a carbon dioxide-rich feed, a steam feed, a saturated hydrocarbon feed, a hydrogen-rich feed, or a combination thereof.

The dilution feed should have an upper limit for the olefin content. Suitably, the dilution feed should comprise less than 0.5 mol %, preferably less than 0.2 mol %, more preferably less than 0.1 mol % olefins.

When steam is used as a dilution feed, there is a limit. Steam may only be added to the extent that the inlet to the conversion stage is above the dew point of the combined feed, so that water does not condense.

In one preferred aspect, there is no external dilution feed. The dilution feed is—in this aspect—one or more feeds selected from:
a second portion of said first feed of carbon dioxide, and
a second portion of said second feed of hydrogen.

Preferably, in this aspect, the dilution feed is the second portion of the first feed of CO₂.

The present invention allows the exotherm across an adiabatic hydrogenation reactor catalyst bed to be controlled efficiently without employing more complicated reactor design and/or additional equipment. This is achieved by introducing dilution gas to the reactant off-gas containing unsaturated hydrocarbons. The dilution gas should ideally be such that it doesn't participate in the hydrogenation reaction but functions as the sink to the heat generated from exothermic hydrogenation reaction.

An effective dilution gas could be a carbon dioxide (CO₂) rich gas stream or a methane (CH₄) rich gas stream or steam, or a mixture of these in any ratio. Preferably, the dilution gas should not influence downstream processes or unit operations. For example, at least a part of CO₂ rich gas stream, can be added to the unsaturated hydrocarbon containing off-gas stream for controlling the exotherm in adiabatic hydrogenator. At least a part of CO₂ rich feed, which acts as the dilution gas in the hydrogenation, gets reacted in downstream syngas generation unit in presence of H₂ rich feed. Thus, using at least a part of CO₂ rich feed stream as dilution gas for hydrogenation of unsaturated hydrocarbon containing off-gas feed in eFuels or eChemical plant provides a good synergy.

In another option, in a hybrid eFuels or eChemicals plant (i.e. a plant that uses both sustainable and fossil-based feed sources) at least a part of the methane (CH₄) rich gas stream can be used as the dilution gas for hydrogenation of unsaturated hydrocarbon containing off-gas stream. Alternatively, mix of at least a part of CO₂ rich feed stream and at least a part of the methane (CH₄) rich gas stream can also be used as dilution gas for the same purpose.

In a preferred aspect, the dilution feed is a carbon dioxide-rich feed where carbon dioxide-rich means that the major portion of this feed is CO₂; i.e. over 75%, such as over 85%, preferably over 90%, more preferably over 95%, even more preferably over 99% of the dilution feed is CO₂. Suitably, the dilution feed consists essentially of CO₂. In a preferred aspect, said dilution feed is a carbon dioxide-rich feed, and is preferably a second portion of said first feed of carbon dioxide. Dependent on the system layout, a preferred aspect may also be that said dilution feed is a carbon dioxide-rich feed provided as an external dilution feed. The advantage of a dilution feed being a carbon dioxide-rich feed is that carbon dioxide is comprised in the feed to react further down the process in the syngas stage. In this way, adding a dilution feed being a carbon dioxide-rich feed provides a perfect synergy to the process.

In another—less preferred—aspect, the dilution feed is a hydrogen-rich feed where hydrogen-rich means that the major portion of this feed is hydrogen; i.e. over 75%, such as over 85%, preferably over 90%, more preferably over 95%, even more preferably over 99% of this feed is hydrogen. Suitably, the dilution feed consists essentially of hydrogen. In one aspect, said dilution feed is a hydrogen-rich feed, and is preferably a second portion of said second feed of hydrogen. Dependent on the system layout, a preferred aspect may also be that said dilution feed is a hydrogen-rich feed provided as an external dilution feed. The advantage of a dilution feed being a hydrogen-rich feed is that hydrogen is comprised in the feed to react further down the process in the syngas stage. However, including a hydrogen-rich dilution feed will increase the hydrogen consumption of the system and thereby the cost of hydrogen production.

In one aspect, the dilution feed is a saturated hydrocarbon feed, such as a methane-rich feed A “saturated hydrocarbon feed” is a feed of saturated hydrocarbons, meaning that the major portion of the dilution feed is saturated hydrocarbons; i.e. over 75%, such as over 85%, preferably over 90%, more preferably over 95%, even more preferably over 99% of the dilution feed is saturated hydrocarbons. “Methane-rich” means that the major portion of the dilution feed is methane; i.e. over 75%, such as over 85%, preferably over 90%, more preferably over 95%, even more preferably over 99% of the dilution feed is methane. In aspects where the syngas stage comprises a non-selective RWGS unit such that one or more of the methanation reactions (including also reverse methanation i.e. steam reforming) takes place in addition to reverse water gas shift, the advantage of a dilution feed being a saturated hydrocarbon feed is that it can be converted in downstream processes in the presence of appropriate amount of steam addition and thus, provides a synergy in so-called hybrid type eFuels or eChemical plants. Notably, in this case, the saturated hydrocarbon feed should not comprise any unsaturated hydrocarbons, e.g. it should comprise less than 0.5 mol %, preferably less than 0.2 mol %, more preferably less than 0.1 mol % olefins.

In aspects comprising a reforming unit, which includes an ATR, the methane can act as a reactant within steam reforming. However, including a methane-rich dilution feed will increase the overall hydrocarbon consumption of the system, thereby lowering the sustainability and efficiency of the system.

In another aspect, the dilution feed comprises a second portion of the first feed of carbon dioxide, and a second portion of said second feed of hydrogen.

The amount of dilution feed may be regulated such that the mixing means receives dilution feed to off-gas sufficient to regulate the exotherm in the subsequent hydrogenation unit. It may be possible to e.g. introduce less dilution feed during start-up, before strong exotherm is generated, and then increase the dilution feed during full operation.

### Off-Gas Conversion Stage

An off-gas conversion stage is arranged to receive at least a portion of the combined feed and provide a pre-converted off-gas stream to the syngas stage. The off-gas conversion stage comprises at least one hydrogenation unit and is arranged to convert most or all unsaturated hydrocarbons in said combined feed to saturated hydrocarbons in said pre-converted off-gas stream.

The hydrogenation unit converts the olefins present in the off-gas into alkanes through a hydrogenation process. The hydrogenation process occurs in the hydrogenation reactor and is facilitated by a hydrogenation catalyst, which operates with minimum possible inlet temperature. The effluent gas from the hydrogenation reactor comprises carbon monoxide, carbon dioxide, hydrogen, methane and higher hydrocarbons. Such a gas could for example contain carbon monoxide (10-30 vol. %), hydrogen (10-30% vol %), carbon dioxide (20-70 vol %), methane (5-25 vol %) and higher hydrocarbons (0.2-10 vol %).

In some cases, and depending upon the composition of the off-gas stream, a hydrogenation outlet temperature above 200° C. may initiate other exothermic side reactions (such as methanol or higher alcohol formation) causing inefficient conversion of unsaturated hydrocarbons and potential cracking of unconverted unsaturated hydrocarbons and thereby, carbon formation. Therefore, it is preferred to keep the hydrogenation outlet temperature below 200° C., preferably below 180° C. In the present system, the off-gas conversion stage is arranged to have an outlet temperature of no more than 200° C. degrees. The hydrogenated off-gas temperature is kept within the temperature limit by controlling the ratio of dilution feed to off-gas added to the mixing means. This ratio depends on the unsaturated hydrocarbon content in the off-gas stream. In this way, the present system provides efficient control of exotherm from the hydrogenation.

In one aspect of the system of the invention, the combined feed is provided to the off-gas conversion stage at a temperature of 80-200° C., and the off-gas conversion stage is arranged to provide the pre-converted off-gas stream at a temperature of 80-200° C., preferably 90-180° C. The system may further comprise regulating means for adjusting the dilution feed to ensure an exotherm across the hydrogenation unit which is less than 120° C., preferably less than 80° C., most preferably less than 60° C. Suitable regulating means are those which regulate the volume, temperature or rate of addition of the dilution feed.

Before routing the hydrogenated off-gas to the syngas stage, the effluent from the hydrogenation reactor may also be directed to a higher hydrocarbon conversion reactor. This higher hydrocarbon conversion reactor may be adiabatic or cooled and the catalyst will typically be pellet-based. In this higher hydrocarbon conversion reactor, the RWGS reaction (1) (or the shift reaction, which is the reverse of (1)) and methanation reactions (2)-(3) or the reverse methanation reactions (depending upon the gas composition, temperature, and pressure) take place. Furthermore, steam reforming of higher hydrocarbons may take place in this reactor:

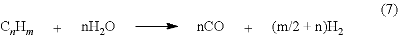

The conditions of the reactor are preferably adjusted to convert more than 90%, such as more than 95%, such as more than 99% of the non-methane hydrocarbons present in the feed mixture. Removal or substantial reduction of non-methane hydrocarbons has the advantage that the risk of carbon formation in the syngas generation unit such as in e-RWGS reactor(s) is reduced considerably.

The exit temperature from this higher hydrocarbon conversion reactor is typically in the range between 300-700° C. The effluent from this reactor, which is the hydrogenated F-T tail gas, is fed to the syngas stage optionally after cooling and removal of condensate. This has the advantage that the amount of CO₂ in the effluent from the syngas stage will be lower. The hydrogenated F-T tail gas provided from the higher hydrocarbon conversion reactor may be mixed with the first feed and the second feed before being fed to the syngas stage.

Alternatively, the hydrogenated off-gas stream is first passed through a water gas shift reactor together with steam (reverse of reaction 1) before sending it to the higher hydrocarbon conversion reactor. This reduces the CO-concentration at the inlet to the syngas stage reducing the potential for carbon formation c.f. reactions (4)-(5).

In the simplest aspect where the syngas stage comprises a stand-alone RWGS unit, the olefins provided in the off-gas may result in carbon formation. Therefore, the F-T tail gas is arranged to be hydrogenated followed by removal of higher hydrocarbons. This is advantageous as the catalyst for higher hydrocarbon conversion is similar in nature to non-selective RWGS catalysts.

In an alternative aspect, the off-gas conversion stage comprises a reforming unit, which suitably includes one or more ATR reactor(s). Within this aspect, higher hydrocarbons provided in the F-T tail gas may undergo partial combustion followed by steam reforming of the partially combusted hydrocarbons. Thus, the higher hydrocarbons can be processed in the ATR reactor(s) wherefore the hydrocarbons are not removed before being fed to the syngas stage. However, as saturated hydrocarbons undergo a more complete combustion process than olefins, it is preferred to hydrogenate the F-T tail gas before feeding the F-T tail gas as a feed to the syngas stage. An advantage of hydrogenation is thus a decrease in the amount of carbon formation formed within the syngas stage. U.S. Pat. No. 9,161,886B2 concerns the use of hydrogenated FT tail gas for ATR.

In another aspect, the syngas stage comprises a RWGS unit and a reforming unit, which suitably includes one or more ATR reactor(s). Within this aspect, the hydrogenated tail gas will typically not go through the higher hydrocarbon conversion reactor and the will preferably be fed to the syngas stage between the RWGS unit and the reforming unit.

### Optional Stages

In one aspect, the system comprises a feed purification stage. The feed purification stage is arranged to provide a said first portion of carbon dioxide feed and/or said first portion of said hydrogen feed or an admixture of the two first portions to the syngas stage and, optionally, to provide the dilution feed comprising a second portion of said first feed of carbon dioxide or a second portion of said second feed of hydrogen to the mixing means.

### Product Work-Up Stage

In one aspect, the system comprises a product work-up stage. The product work-up stage is arranged to receive the product stream from the synthesis stage, and provide a processed (i.e. upgraded) product stream.

When the synthesis stage is an FT synthesis stage, the product work-up stage is arranged to receive the hydrocarbon product stream and to provide a processed hydrocarbon stream.

In this case, the product work-up stage comprises a hydroprocessing and hydrocracking unit. In one aspect, the primary product(s) from F-T synthesis stage is/are typically jet fuel and/or kerosene (e.g. comprising primarily C12-C15) such as synthetic paraffinic kerosene (SPK) and/or diesel (e.g. comprising primarily C15-C20). Besides, naphtha (e.g. comprising primarily C5-C12) and Liquified Petroleum Gas (LPG; e.g. comprising primarily C3-C4) streams are also produced in F-T synthesis stage. In one aspect, product streams may also be recycled to the syngas stage from the F-T synthesis stage.

### Processes

A process is provided for the production of a product stream in a system as described herein, said process comprising the steps of:
providing a first feed comprising carbon dioxide and a second feed comprising hydrogen, and optionally an external dilution feed;
feeding at least a first portion of said carbon dioxide feed, at least a first portion of said hydrogen feed and at least a portion of a pre-converted off-gas stream, to the syngas stage to provide a first synthesis gas stream;
feeding at least a portion of the first synthesis gas stream to the synthesis stage to provide one or more product streams and an off-gas stream comprising unsaturated hydrocarbons;
combining at least a portion of the off-gas stream with at least one dilution feed using a mixing means to provide a combined feed;
feeding at least a portion of the combined feed to the off-gas conversion stage and converting unsaturated hydrocarbons in said combined feed to saturated hydrocarbons in said pre-converted off-gas stream; to provide a pre-converted off-gas stream to the syngas stage;
   and wherein the dilution feed is selected from:
a second portion of said first feed of carbon dioxide,
a second portion of said second feed of hydrogen,
an external dilution feed (3), comprising a carbon dioxide-rich feed, a steam feed, a saturated hydrocarbon feed, a hydrogen-rich feed, or a combination thereof.

In one aspect of the process, the process comprises the step of feeding the converted off-gas stream from the off-gas conversion stage to the syngas stage in admixture with said first and or second feeds. In one aspect of the process, said first and second feeds are fed to the syngas stage as an admixed feed.

In one preferred aspect of the process, the dilution feed is a carbon dioxide-rich feed, preferably a second portion of said first feed of carbon dioxide. The advantage of using a carbon dioxide-rich feed as the dilution feed is that carbon dioxide is comprised in the reaction further down the process in the syngas stage. In this way, adding a dilution feed being a carbon dioxide-rich feed provides a perfect synergy to the process.

In one aspect of the process, the mixing means and dilution feed are regulated, such that a sufficiently low temperature of the output from the hydrogenation unit is achieved. Thus, in one aspect, the pre-converted off-gas stream at the outlet of the off-gas conversion stage has a temperature of no more than 200° C. degrees.

Suitably, the combined feed is provided to the off-gas conversion stage at a temperature of 80-200° C., and the off-gas conversion stage provides the pre-converted off-gas stream at a temperature of 80-200° C., preferably 90-180° C. In a preferred aspect, the dilution feed is adjusted to ensure an exotherm across the hydrogenation unit which is less than 120° C., preferably less than 80° C., most preferably less than 60° C.

In one aspect, the system comprises a feed purification stage. Within this system, it is a preferred aspect of the process, is that the process further comprises a step of providing a said first portion of carbon dioxide feed and/or said first portion of said hydrogen feed or an admixture of the two first portions from the feed purification stage to the syngas stage and, optionally, providing a dilution feed comprising a second portion of said first feed of carbon dioxide or a second portion of said second feed of hydrogen to the mixing means.

In one aspect, the system comprises a product work-up stage. Within this system, it is a preferred aspect of the process, that said process comprises the step of feeding said product stream to the product work-up stage and providing a processed product stream.

### Specific Embodiments

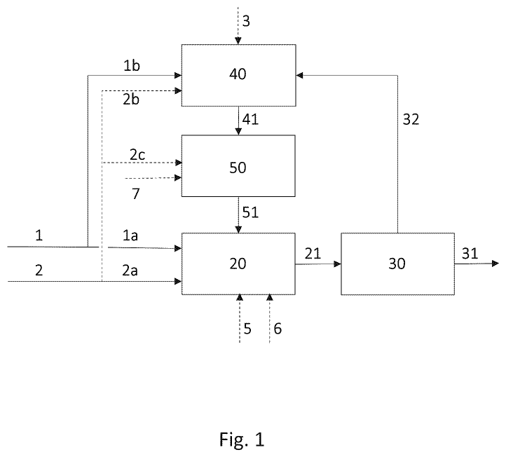

FIG. 1 shows a first layout of the system. The system comprises a syngas stage (20), a synthesis stage (30), mixing means (40), and an off-gas conversion stage (50) from which a pre-converted off-gas stream (51) is recycled into the syngas stage (20). The system feeds in FIG. 1 are as follows:
first portion (1a) of first feed (1) comprising carbon dioxide to the syngas stage (20);
first portion (2a) of second feed (2) comprising hydrogen to the syngas stage (20);
dilution feed (1b, 2b, 3) to the mixing means (40).

First portion (1a) of first feed (1) comprising carbon dioxide and first portion (2a) of second feed (2) comprising hydrogen are supplied to the syngas stage (20), which converts them, together with pre-converted off-gas stream (51) to a first synthesis gas stream (21). The first synthesis gas stream (21) is obtained after removal of process condensate in the syngas stage (not shown in the figure). The first synthesis gas stream (21) is then fed to the synthesis stage (30) where it is converted to at least a product stream (31) and an off-gas stream (32). The off-gas stream (32) is fed to mixing means (40) wherein it is mixed with a dilution feed (1b and optionally 2b and/or optionally,3) to provide a combined feed (41).

The combined feed (41) is fed to the off-gas conversion stage (50) which converts the combined feed (41) to a pre-converted off-gas stream (51) which is then fed as a feed to the syngas stage (20). The pre-converted off-gas stream (51) can be obtained after removal of process condensate in off-gas conversion stage (50) (not shown in the figure).

The off-gas conversion stage (50) is a stage comprising multiple reactors. The off-gas conversion stage (50) comprises—preferably—not only hydrogenator, but also water gas shift reactor and higher hydrocarbon removal reactor. If needed, a part of second feed (2c) comprising hydrogen can optionally be fed to the off-gas conversion stage (50). When WGS and/or removal of higher hydrocarbon is opted, steam (7) is added to the off-gas conversion stage (50). Optionally, an external hydrocarbon feed (6) (such as natural gas) can be fed to syngas stage (20).

Fifth feed (5) comprising an oxidant may be fed to the syngas stage (20), when syngas stage comprises autothermal reactor (ATR) section. Optionally, a hydrocarbon feed (6) (such as natural gas) can be fed to syngas stage (20).

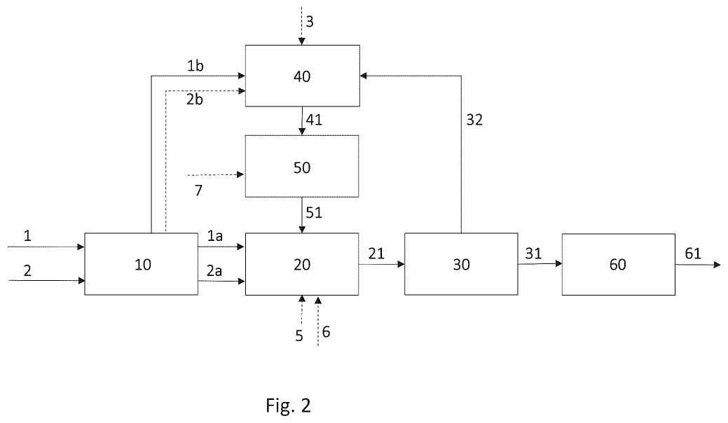

FIG. 2 shows a more developed layout of the system. Within this layout the first feed (1) comprising carbon dioxide and the second feed (2) comprising hydrogen is fed to a purification stage (10) before a first portion (1a) of said first feed comprising carbon dioxide and a first portion (2a) of said second feed comprising hydrogen are supplied to the syngas stage (20). The purification stage (10) provides a second portion (1b) of said first feed (1) of carbon dioxide and optionally, second portion (2b) of said second feed (2) of hydrogen to the mixing means (40). In this layout, also a product work-up stage (60) is included such that the product work-up stage (60) is fed with the product stream (31) provided by the synthesis stage (30) and provides a processed product stream (61). The product work-up stage (60) normally needs a feed of hydrogen (not shown).

### Example 1

A set of calculations is performed for a system such as an eFuel plant (i.e. production of jet-fuel, kerosene, diesel etc. from CO₂ and H₂ feed). All calculations are based on the same amount of end-product with similar recycled tail gas from a downstream Fischer-Tropsch (F-T) synthesis stage. In these examples, the tail gas contains ca. 7 mol % olefin (propylene) along with other constituents (e.g. H₂, CO, CO₂, CH₄, C2+ alkanes, oxygenates etc.).

Using recycled tail gas as feed in the syngas stage results in better utilization of feeds. However, it requires pre-conversion of tail gas before sending them to the RWGS reactor to avoid carbon formation. Pre-conversion of tail gas comprises hydrogenation of unsaturated hydrocarbons (here propylene), present in the F-T tail gas. From prior art, maximum exotherm in hydrogenation reactor should be kept within ca.100° C. to avoid carbon formation in the hydrogenator and potentially also in downstream reactor(s).

| Parameters | UoM | Case1 | Case2 | Case3 |
|---|---|---|---|---|
| Fresh CO₂ feed | % | 100 | 100 | 100 |
| Fresh H₂ feed | % | 100 | 100 | 100 |
| H₂/CO₂ in fresh feed | mol/mol | 2.91 | 2.91 | 2.90 |
| Olefin content in tail gas | mol % | 7.00 | 7.00 | 7.00 |
| Olefin hydrogenator Tin | ° C. | 100 | 98 | 98 |
| Olefin hydrogenator Tout | ° C. | 307 | 200 | 200 |
| Recycle of hydrogenator effluent | % | 0 | 53 | 0 |
| Hydrogenator effluent recycle | — | No | Yes | No |
| cooler | | | | |
| Hydrogenator effluent recycle | — | No | Yes | No |
| compressor | | | | |
| Part of fresh CO₂ feed to | % | 0 | 0 | 22 |
| hydrogenator | | | | |

In case 1, recycled F-T tail gas feed is sent directly to the hydrogenator without any means for control of exotherm across the hydrogenator. As a result, with available tail gas composition, the exotherm across the hydrogenator is much higher than the limit of ca. 100° C., causing formation of by-products and inefficient olefin conversion, which ultimately would result in carbon formation.

In case 2, control of exotherm and thereby, avoidance of carbon formation is achieved by recycling ca. 55% of the hydrogenator effluent back to the hydrogenator inlet. This solution, however, requires additional equipment, such as a cooler and compressor/blower for effluent recycle.

In case 3, exotherm across the hydrogenator is controlled without using any additional equipment, as in case 2. It is done by routing approx. 22% of fresh total CO₂ feed as a dilution feed to the recycle F-T tail gas feed. This additional CO₂ acts as heat sink in the hydrogenation reaction, resulting in limited exotherm without carbon formation. This is particularly advantageous for such systems, because added CO₂, inert in the hydrogenation reaction, gets consumed as feed via RWGS in subsequent steps in syngas stage. It's also important to notice that effectively there is no change in fresh CO₂ and H₂ feed consumptions in either of the solutions.

The present invention has been described with reference to a number of aspects and figures. However, the skilled person is able to select and combine various aspects within the scope of the invention, which is defined by the appended claims. All documents referenced herein are incorporated by reference.

1. A system for producing a product stream, said system comprising:
a first feed comprising carbon dioxide;
a second feed comprising hydrogen;
optionally, an external dilution feed;
a syngas stage arranged to receive at least a first portion of said first feed, at least a first portion of said second feed, and at least a portion of a pre-converted off-gas stream, and to provide a first synthesis gas stream;
a synthesis stage arranged to receive said first synthesis gas stream, and to provide one or more product stream(s) and an off-gas stream comprising unsaturated hydrocarbons;
mixing means arranged to combine at least a portion of the off-gas stream with a dilution feed and provide a combined feed;
an off-gas conversion stage arranged to receive at least a portion of the combined feed and provide a pre-converted off-gas stream to the syngas stage;
wherein the off-gas conversion stage comprises at least one hydrogenation unit arranged to convert unsaturated hydrocarbons in said combined feed to saturated hydrocarbons in said pre-converted off-gas stream;
and wherein the dilution feed is one or more feeds selected from:
a second portion of said first feed of carbon dioxide,
a second portion of said second feed of hydrogen, and
the external dilution feed, comprising a carbon dioxide-rich feed, a steam feed, a saturated hydrocarbon feed, a hydrogen-rich feed, or a combination thereof.

2. The system according to claim 1, wherein the unsaturated hydrocarbons in the off-gas stream comprise alkenes (CₙH₂ₙ; n≥2).

3. The system according to claim 1, wherein the hydrogenation unit comprises at least an adiabatic reactor comprising a catalyst, wherein no effluent is arranged to be recycled or no indirect exchange of heat is applied for control of exotherm across the catalyst bed.

4. The system according to claim 1, further comprising regulating means for adjusting the dilution feed to ensure an exotherm across the hydrogenation unit which is less than 120° C.

5. The system according to claim 1, wherein the off-gas conversion stage further comprises a water gas shift unit downstream said hydrogenation unit and a higher hydrocarbon conversion unit downstream said water gas shift unit; said system further comprising a steam feed to said water gas shift unit.

6. The system according to claim 1, wherein the off-gas conversion stage is arranged to separate a condensate stream from the pre-converted off-gas stream, downstream the higher hydrocarbon removal unit.

7. The system according to claim 1, wherein the syngas stage comprises a reverse water gas shift stage.

8. The system according to claim 1, wherein the syngas stage comprises an autothermal reactor unit and wherein an external feed of an oxidant.

9. The system according to claim 1, wherein the system further comprises an external hydrocarbon feed, arranged to be fed to said syngas stage, wherein the ratio of moles of carbon in said hydrocarbon feed to the moles of carbon in carbon-dioxide in said first portion of said first feed is less than 0.5.

10. The system according to claim 1, wherein the synthesis stage comprises a Fischer-Tropsch synthesis stage.

11. The system according to claim 1, wherein the synthesis stage comprises a methanol synthesis stage followed by an olefin synthesis stage and/or a kerosene or diesel synthesis stage.

12. The system according to claim 1, wherein said dilution feed is a carbon dioxide-rich feed.

13. The system according to claim 1, wherein said dilution feed comprises a second portion of said first feed of carbon dioxide, and optionally, a second portion of said second feed of hydrogen.

14. The system according to claim 1, wherein the pre-converted off-gas stream from the off-gas conversion stage is arranged to be fed to the syngas stage in admixture with said first portion of said first feed and/or second portion of said second feed.

15. A process for production of a product stream in a system according to claim 1, said process comprising the steps of:
providing the first feed comprising carbon dioxide, the second feed comprising hydrogen, and optionally the external dilution feed;
feeding at least the first portion of said first feed, at least the first portion of said second feed and at least the portion of a pre-converted off-gas stream, to the syngas stage to provide the first synthesis gas stream;
feeding the at least a portion of the first synthesis gas stream to the synthesis stage to provide the one or more product stream(s) and the off-gas stream comprising unsaturated hydrocarbons;
combining the at least a portion of the off-gas stream with the dilution feed in said mixing means to provide the combined feed;
feeding the at least a portion of the combined feed to the off-gas conversion stage to provide the pre-converted off-gas stream to the syngas stage; and
converting unsaturated hydrocarbons in said combined feed to saturated hydrocarbons in said pre-converted off-gas stream;
and wherein the dilution feed is the one or more feeds selected from:
the second portion of said first feed of carbon dioxide,
the second portion of said second feed of hydrogen, and
the external dilution feed, comprising the carbon dioxide-rich feed, the steam feed, the saturated hydrocarbon feed, the hydrogen-rich feed, or the combination thereof.

16. The process according to claim 15, wherein said process comprises the step of feeding the converted off-gas stream from the off-gas conversion stage to the syngas stage in admixture with said first and/or second feeds.

17. The process according to claim 15, wherein the dilution feed is a carbon dioxide-rich feed.

18. The process according to claim 16, wherein the dilution feed is adjusted to ensure an exotherm across the hydrogenation unit which is less than 120° C.

19. The system according to claim 1, wherein the dilution feed is the second portion of said first feed of carbon dioxide and/or the second portion of said second feed of hydrogen.

20. The system according to claim 1, wherein the system comprises the external dilution feed, and the dilution feed includes the external dilution feed.
